(19) United States
(12) Patent Application Publication
(10) Pub. No.: US 2005/0026239 A1
(43) Pub. Date: Feb. 3, 2005

(54) Kit for detecting endotoxin in aqueous solutions

(75) Inventors: Carlos A. Castro, Cotuit, MA (US); Richard J. Ridge, Falmouth, MA (US); Thomas J. Novitsky, East Falmouth, MA (US)

Correspondence Address: LOWRIE, LANDO & ANASTASI, RIVERFRONT OFFICE, ONE MAIN STREET, ELEVENTH FLOOR, CAMBRIDGE, MA, 02142, US

(73) Assignee: Associates of Cape Cod, Inc., East Falmouth, MA (US)

(21) Appl. No.: 10/826,922

(22) Filed: Apr. 19, 2004

Related U.S. Application Data
(60) Provisional application No. 60/463,737, filed on Apr. 18, 2003.

(51) Int. Cl. C12Q 1/04

(52) U.S. Cl. 435/34

(57) ABSTRACT

The present invention relates to a simple, rapid, and cost-effective test kit for specifically detecting bacterial endotoxin in aqueous solutions, such as water or dialysate solutions, using a Limulus Amebocyte Lysate (LAL)-based gel clot assay. Advantageously, the test kit can vary in its level of sensitivity for detecting endotoxin.

### CROSS-REFERENCE TO RELATED APPLICATIONS

This application claims the priority benefit of U.S. Provisional Application No. 60/464,737, filed Apr. 18, 2003, the content of which is incorporated by reference in its entirety.

### BACKGROUND OF THE INVENTION

1. Field of the Invention

This invention relates to a simple, rapid, and cost-effective test kit for specifically detecting bacterial endotoxin in aqueous solutions, such as water or dialysate, using a Limulus Amebocyte Lysate (LAL)-based gel clot assay.

2. Related Art

Bacterial endotoxins, also known as pyrogens, are the fever-producing byproducts of Gram-negative bacteria and can be dangerous or even deadly to humans. Symptoms of infection and presence of endotoxin range from fever, in mild cases, to death.

Cells from the hemolymph of the horseshoe crab (amebocytes) contain an endotoxin-binding protein (Factor C) that initiates a series of complex enzymatic reactions resulting in clot formation when the cells are in contact with endotoxin (reviewed in Iwanaga, Curr. Opin. Immunol. 5: 74-82 (1993)). The endotoxin-mediated activation of an extract of these cells, i.e., amebocyte lysate, is well-understood and has been thoroughly documented in the art.

See, for example, Levin et al., Thromb. Diath. Haemorrh. 19: 186-197(1968); Nakamura et al., Eur. J. Biochem. 154: 511-521 (1986); Muta et al., J. Biochem. 101: 1321-1330 (1987); and Ho et al., Biochem. Mol. Biol. Int. 29: 687-694 (1993). This phenomenon has been exploited in bioassays to detect endotoxin in a variety of test samples, including human and animal pharmaceuticals, biological products, research products, and medical devices.

The horseshoe crab Limulus polyphemus is particularly sensitive to endotoxin. Accordingly, the blood cells from this horseshoe crab, termed “Limulus amebocyte lysate” or “LAL,” are employed widely in endotoxin assays of choice because of their sensitivity, specificity, and relative ease for avoiding interference by other components that may be present in a sample. See, e.g., U.S. Pat. Nos. 4,495,294, 4,276,050, 4,273,557, 4,221,865, and 4,221,866. LAL, when combined with a sample containing bacterial endotoxin, reacts with the endotoxin to produce a product, for example, a gel clot or chromogenic product, that can be detected, for example, either visually, or by the use of an optical detector.

Although the enzymatic clotting cascade of LAL initially was considered specific for endotoxin, it was later discovered that β-(1,3)-D-glucans also activate the clotting cascade of LAL through a partially shared pathway, referred to as the Factor G pathway. See, for example, Morita et al., FEBS Lett. 129: 318-321 (1981); and Iwanaga et al, J. Protein Chem. 5: 255-268 (1986). Accordingly, if a sufficient amount of β-(1,3)-D-glucans are present in a sample, a LAL positive response may occur that is independent of the endotoxin-mediated response. Thus, it has become very important to increase the specificity of LAL for endotoxin, i.e., by utilizing an endotoxin-specific amebocyte lysate preparation.

In one approach for achieving endotoxin-specificity of amebocyte lysate, polysaccharide based Factor G inhibitors are combined with amebocyte lysate to reduce or eliminate clotting induced by β-(1,3)-D-glucans present in the biological sample. See, for example, U.S. Pat. Nos. 5,155,032; 5,474,984; and 5,641,643.

Other approaches are known by those skilled in the art for increasing the specificity of LAL for endotoxins. For example, U.S. Pat. No. 5,401,647 discloses a method for removing Factor G from LAL by combining LAL with β-(1,3)-D-glucans immobilized on an insoluble carrier. Once bound to the carrier via the β-(1,3)-D-glucan moiety, the Factor G can thereafter be removed from the LAL to produce a Factor G depleted lysate. Similarly, U.S. Pat. No. 5,605,806 discloses an immunoaffinity based method using a Factor G specific antibody to remove Factor G from LAL thereby to produce a Factor G depleted amebocyte lysate. Finally, Kakinuma, A. et al. describe a method that employs the addition of excess glucan to the lysate to overwhelm Factor G and prevent additional glucan from activating the lysate. See, Kakinuma, A. et al., Biochem. Biophys. Res. Commun. 101:434-439 (1981).

Endotoxins are a significant concern in the field of nephrology. About 300,000 individuals in the U.S. receive some form of dialysis, which provides life-saving renal replacement for end-stage renal disease (ESRD). Water for dialysis as well as dialysates are not sterile, and can contain significant concentrations of bacteria and endotoxins. Hemodialysis is a water-intensive therapy that presents an enormous challenge to produce copious amounts of high purity water, cost effectively.

In a typical dialysis system, blood and dialysate are pumped into the dialyzer (also known as the artificial kidney) from opposite directions. If the hydrostatic pressure on the dialysate side of the dialysis membrane exceeds the pressure on the blood side, it is possible to transfer endotoxins from the dialysate into the blood (back-filtration). In addition, endotoxins adsorbed to the membrane surface, resulting from a manufacturing error or deposited during a previous use, may be dislodged when the artificial kidney is initially primed with dialysate.

The occurrence of endotoxin-mediated pyrogenic reactions continues to challenge those responsible for dialysis facilities. The potential for exposure of dialysis patients to greater levels of microbial and endotoxin contamination has increased dramatically during the last decade with the increase in reuse of hemodialyzers, and the use of bicarbonate dialysate and high flux dialysis. See, Bland, L.A., Adv Ren Replace Ther: 2:70-79 (1995).

There are significant reasons to reduce the exposure of hemodialysis patients to endotoxins. The most acute is obviously to eliminate pyrogenic reactions. However, even more critical are the well-documented effects of long-term exposure to pyrogens, including leukocyte and monocyte activation, platelet activation, increased adhesiveness and aggregation, and complement activation, which together with hyperlipidemia, cause endothelium damage and lipid deposition in the arterial wall. Therefore, it is expected that regular use of sterile and endotoxin-free dialysate will help decrease the cardiovascular morbidity and mortality rate of patients undergoing hemodialysis. See, e.g., Amato, R.L., Nephrology Nursing Journal 28: 619-629 (2001).

Chronic inflammatory responses due to long-term consequences of cell stimulation and the subsequent release of inflammatory mediators such as tumor necrosis factor (TNF) and interleukin-1 (IL-1) are a major concern as well. See, Canaud, B., et al., “Microbiologic Purity of Dialysate:Rationale and Technical Aspects,” in Chronic Inflammation in Hemodialysis, pp. 34-47, Switzerland: S. Karger AG (2000). The use of sterile and endotoxin-free dialysate significantly decreases the interleukin levels in patients' blood.

Dialysis amyloidosis is considered an inflammatory disease; the major protein of amyloid deposits is beta-2-microglobulin. Synthesis of beta-2microglobulin in macrophages is enhanced by endotoxins. Therefore, dialysis water contaminated with endotoxin may contribute to this process. Bad et al. showed that the onset of amyloidosis in long-term dialysis patients was considerably delayed when ultrapure dialysate was used (Bad, M. et al.: Int. J. Artif. Organs 14:681-685 (1991)).

To help prevent pyrogenic reactions and bacteremia in hemodialysis patients caused by microbial and endotoxin contamination of hemodialysis fluids, the Association for the Advancement of Medical Instrumentation (AAMI) has recently promulgated standards for maximum allowable concentrations of bacteria and endotoxin in these fluids (endotoxin level should not exceed 2.0 EU/mL as tested by the LAL assay, and action must be taken when the level exceeds 1.0 EU/mL); see, Association for the Advancement of Medical Instrumentation (AAMI), Vol. 3: Hemodialysis systems; ANSI/AAMI, RD62-2001, Arlington, Va. (2001).

It has been recommended that each dialysis center develop microbiological and endotoxin surveillance policies and procedures for the types of hemodialysis fluids to assay, frequency and manner of sample collection, assay techniques, and methods for recording and interpreting results to ensure compliance with the AAMI standards. Clearly, a safer environment would be provided for each dialysis patient if appropriate microbiological assay procedures are followed and the results are consistently within the AAMI microbiological and endotoxin standards.

Currently, available LAL tests on dialysate rely on one of three methods: The first is a standard gel-clot assay. This assay takes 60 minutes and requires the user to “select” a sensitivity, which is unique to a particular lot of LAL. If the user wants to have control over the sensitivity or shorten the assay time (<60 minutes), they need to use one of the two photometric LAL methods, either the turbidimetric or the kinetic chromogenic method. Both of these methods, however, require specialized technical expertise and a machine to read the test, e.g., a microplate reader. Further, photometric LAL methods are expensive.

Clearly, there is a need in the art for a rapid, simple, and cost-effective test kit for specifically detecting endotoxin in aqueous solutions such as water and dialysate solutions, that would combine the ease of use of a gel-clot LAL assay with the speed and multi-sensitivity of the photometric methods, but without requiring specialized equipment or expertise. This need is particularly felt in the renal dialysis clinic.

### BRIEF SUMMARY OF THE INVENTION

It is an object of the invention to provide a simple, rapid, multi-sensitive, and cost-effective gel clot assay for specifically detecting endotoxin in aqueous solutions, such as water or dialysate solutions. This object of the invention is provided by one or more of the embodiments described below.

One embodiment of the invention provides a test kit for specifically detecting endotoxin in aqueous solutions. The test kit comprises, at a minimum: (a) at least one first container containing freeze dried, endotoxin-specific, horseshoe crab amebocyte lysate, whereby the sensitivity of the lysate is pre-certified; (b) at least one second container containing a defined quantity of endotoxin to serve as a positive control, wherein said defined quantity of endotoxin is pre-certified to positively react with the amebocyte lysate present in said first container; and (c) at least one disposable endotoxin-free transfer instrument. The sensitivity of the test kit (i.e., the amount of endotoxin (EU) the kit can detect) can vary based on two factors: (1) the time of incubation of the test; and (2) the formulation of the lysate in the first container. Also, the ability of the test kit to indicate the absence of sample interference or inhibition, i.e., provide a valid test result, is based on the quantity of endotoxin contained in the positive control.

The gel-clot LAL assay described herein is especially useful in the kidney dialysis clinic. During kidney dialysis, blood is circulated through a machine which contains a dialyzer. The dialyzer has two spaces separated by a thin membrane. Blood passes on one side of the membrane and dialysis fluid passes on the other. The wastes and excess water pass from the blood through the membrane into the dialysis fluid which is then discarded. The cleansed blood is returned to the patient's bloodstream.

The endotoxin-specific LAL test kit described herein may be used to routinely and more frequently test the water systems used to prepare dialysate, flush lines, and prime dialysis machines prior to use by each patient. The LAL test kit described herein may also be used to test the salt solutions (dialysate) used throughout the actual dialysis session.

In a particularly preferred embodiment, the endotoxin-specific horseshoe crab amebocyte lysate in the first container is isolated from Limulus polyphemus. Although the Limulus amebocyte lysate (LAL) is preferred and may be specifically cited when describing other components of the invention, it is emphasized that other horseshoe crab amebocyte lysates known to those skilled in the art may be utilized in the claimed invention.

In another preferred embodiment, the horseshoe crab amebocyte lysate is made endotoxin-specific by using the horseshoe crab amebocyte lysate factor G activation inhibitor in accordance with the teachings in U.S. Pat. Nos. 5,641,643, 5,474,984, or 5,155,032. Each of these patents is incorporated by reference in their entirety.

In another preferred embodiment, the first and second container(s) in the test kit are test tubes. In particularly preferred embodiments, the test tubes are 12×75 mm and are round-bottomed.

The first test tube(s) contains an endotoxin-specific LAL reagent. The amount of endotoxin-specific LAL can range from 0.4 mL, 0.5 mL, to 0.6 mL, but, 0.5 mL of endotoxin-specific LAL is a particularly preferred amount. The amebocyte lysate is in lyophilized form and will be reconstituted during the assay. The sensitivity of the LAL reagent is pre-certified against the United States Pharmacopeia endotoxin standard.

The second test tube(s) comprises a “matched” positive control sample of endotoxin. To understand the concept of a “matched” positive control, one must first look at the endotoxin positive control in a conventional LAL test.

In conventional LAL tests, an endotoxin positive control is typically prepared by diluting a concentrated endotoxin standard to an appropriate concentration, so that when the standard is added to the sample, a concentration of 2× the sensitivity of the LAL being used (i.e., 2×lambda) results, where lambda is the sensitivity of the LAL. In the conventional test, the addition of the endotoxin to the sample to make the positive control results in a slight dilution, which can adversely affect the outcome of the test. In addition to the dilution effect, the preparation of this positive control can be extremely variable and depends on the skill of the user and the quality of the accessories, i.e., diluents, tubes, pipettes, etc.

In the present invention, the second test tube in the test kit comprises a defined quantity of endotoxin (Endotoxin Units, EU) that is pre-certified to be exactly 2×lambda or twice the sensitivity of the lysate, thereby ensuring a positive reaction with the LAL contained in the first test tube. Thus, the term “matched positive control” is intended to mean that the defined amount of endotoxin standard in the second container (i.e., the positive control) has been previously tested and certified to be 2×lambda and to give a positive result when combined with the LAL component of the first tube. Using a pre-certified, matched positive control provides a high degree of assurance of a valid test, i.e., a test that is not inhibited by the test sample. Furthermore, using the present test kit, the user of the kit does not need to run a standard or a negative control since all the components of the kit have been certified. A certificate of analysis attesting to the amebocyte lysate sensitivity, endotoxin concentration of the positive control, and/or the endotoxin-free nature of the transfer instrument may be provided with each test kit. Preferably, the certificate of analysis would be in written form.

The test tubes in the kit and/or the test tube caps may be color-coded and identified by ink-jet or other suitable labels (e.g., “Sample”; “Control”) on the tubes themselves to prevent sample mix-ups.

In another preferred embodiment, the disposable endotoxin-free transfer instruments are transfer pipettes.

The test kit described herein may further comprise written instructions for the user.

### DETAILED DESCRIPTION OF THE PREFERRED EMBODIMENT

The present invention provides a simple, rapid, multi-sensitive, and cost-effective test kit for specifically detecting endotoxin in aqueous solutions, such as water or dialysate solutions. The test kit comprises, at a minimum: (a) at least one first container containing freeze dried, endotoxin-specific, horseshoe crab amebocyte lysate, whereby the sensitivity of the lysate is pre-certified; (b) at least one second container containing a defined quantity of endotoxin to serve as a positive control, wherein said defined quantity of endotoxin is pre-certified to positively react with the amebocyte lysate present in said first container; and (c) at least one disposable endotoxin-free transfer instrument. The sensitivity of the test kit can vary based on the time of incubation of the two containers in the kit. Also, the validity of the test kit is based on the quantity of endotoxin contained in the positive control.

As used herein, the term “at least one” means one or more than one.

The gel-clot LAL assay described herein is particularly useful in the dialysis clinic. The assay may be used to routinely, and thus more frequently, test the water systems used to prepare dialysate, flush lines, and prime dialysis machines prior to use by patients. The LAL assay described herein may also be used to test the salt solutions (dialysate) used in the actual dialysis machine.

The endotoxin-specific horseshoe crab amebocyte lysate used in the first container of the test kit may be isolated from any of the four known species of horseshoe crab: Limulus polyphemus, Tachypleus gigas, Tachypleus tridentatus, or Carcinoscorpius rotundicauda. Particularly preferred is amebocyte lysate isolated from Limulus polyphemus, the horseshoe crab found along the North American coast. Although the Limulus amebocyte lysate (LAL) is preferred and may be specifically cited when describing other components of the invention, it is emphasized that other horseshoe crab amebocyte lysates known to those skilled in the art may be utilized in the claimed invention. The amebocyte lysate is in lyophilized form and will be reconstituted during the assay.

In another particularly preferred embodiment, LAL with enhanced sensitivity to endotoxin is prepared according to the teachings in U.S. Pat. No. 4,107,077 and utilized as the reagent in the first container. This patent is incorporated by reference in its entirety.

Another preferred embodiment includes specific LAL formulations, comprising particular combinations and types of salts and pH buffer. These specific LAL formulations impart functionality to the lysate by overcoming inhibition that may be encountered when testing dialysate and other salt solutions. Preferred and particularly preferred LAL formulations are presented below.

In another particularly preferred embodiment, the horseshoe crab amebocyte lysate is made endotoxin-specific by using the horseshoe crab amebocyte lysate factor G activation inhibitor in accordance with the teachings in U.S. Pat. Nos. 5,641,643, 5,474,984, or 5,155,032. Each of these patents is incorporated by reference in their entirety. Of course, any other technique known to the skilled artisan for making the amebocyte lysate endotoxin-specific is within the scope of the claimed invention. As used herein, the term “endotoxin-specific” is intended to mean that the amebocyte lysate in the first container of the test kit will not react with substances other than bacterial endotoxin, such as, e.g., β-(1,3)-D-glucans, and cause a false positive result.

In another preferred embodiment, the containers found in the test kit are test tubes; 12×75 mm test tubes are particularly preferred as are round-bottomed test tubes. The test tubes in the kit and/or the test tube caps may be color-coded and identified by ink-jet or other suitable labels (e.g., “Sample”; “Control”) on the tubes themselves to prevent sample mix-ups.

The first test tube(s) contains an endotoxin-specific LAL reagent in lyophilized form. The amount of endotoxin-specific LAL can range from 0.4 mL, 0.5 mL, to 0.6 mL, but, 0.5 mL of endotoxin-specific LAL is a particularly preferred quantity. It is believed that 0.5 mL of endotoxin-specific LAL provides sufficient excess reagent to account for pipetting loss during transfer, and thus, is a particularly advantageous quantity of reagent. The sensitivity of the LAL reagent is pre-certified against the United States Pharmacopeia endotoxin standard.

The second test tube(s) comprises a “matched” positive control sample of endotoxin. To understand the concept of a “matched” positive control, one must first look at the endotoxin positive control in a conventional LAL test.

In conventional LAL tests, an endotoxin positive control is typically prepared by diluting a concentrated endotoxin standard to an appropriate concentration, so that when the standard is added to the sample, a concentration of 2× the sensitivity of the LAL being used (i.e., 2×lambda) results, where lambda is the sensitivity of the LAL. In the conventional test, the addition of the endotoxin to the sample to make the positive control results in a slight dilution, which can adversely affect the outcome of the test. In addition to the dilution effect, the preparation of this positive control can be extremely variable and depends on the skill of the user and the quality of the accessories, i.e., diluents, tubes, pipettes, etc.

In the present invention, the second test tube in the test kit comprises a defined quantity of endotoxin (Endotoxin Units, EU) that is pre-certified to be exactly 2×lambda or twice the sensitivity of the lysate, thereby ensuring a positive reaction with the LAL contained in the first test tube. Thus, the term “matched positive control” is intended to mean that the defined amount of endotoxin standard in the second container (i.e., the positive control) has been previously tested and certified to be 2×lambda and to give a positive result when combined with the LAL component of the first tube. Using a pre-certified, matched positive control provides a high degree of assurance of a valid test, i.e., a test that is not inhibited by the test sample. Furthermore, using the present test kit, the user of the kit does not need to run a standard or a negative control since all the components of the kit have been certified.

Accordingly, the test kit may also further comprise a certificate of analysis of the amebocyte lysate sensitivity, the endotoxin concentration of the positive control, and/or the endotoxin-free nature of the transfer instrument (e.g., the pipette). Preferably, the certificate of analysis would be in written form.

In another preferred embodiment, the disposable endotoxin-free transfer instruments are transfer pipettes. Of course, other pipetting devices known to those skilled in the art, e.g., glass or plastic, graduated or volumetric, pipettes and mechanical pipetters with removal tips may be used, so long as they are endotoxin-free. Although endotoxin-free syringes may also be utilized to transfer solutions, most typically, through the sealed cap of the test tube, they are not preferred transfer instruments for several reasons. These reasons include a higher cost, issues relating to disposal and storage, and issues relating to contamination and sterility associated with injection through a test tube stopper.

The test kit described herein may further comprise written instructions for the user.

As used herein, the term “aqueous solution” is intended to mean any sample of purified, distilled, sterile, non-sterile, or filtered water, water for injection, water for irrigation, or reverse osmosis water, or any aqueous solution used in connection with hemodialysis, peritoneal renal dialysis, pre-operative organ perfusion, and/or organ (e.g., renal) transplantation, in which it would be useful to determine possible endotoxin contamination. The term “dialysate” is a preferred example of such an aqueous solution and is intended to mean the salt solutions used in the actual dialysis process. Dialysates can occasionally inhibit an ordinary LAL test (i.e., give false negative). The present invention is designed to overcome inhibition or false negative reactions with all commonly used dialysates. Other aqueous solutions that may be tested, include, e.g., saline and other salt solutions, as well as solutions of sugar, such as dextrose water.

In an ordinary LAL test on dialysate, the tester uses one of three methods. The first is a standard gel-clot assay. This assay takes 60 minutes and requires the user to “select” a sensitivity, which is unique to a particular lot of LAL. If the user wants to have control over the sensitivity or shorten the assay time (<60 minutes), they need to use one of the photometric LAL methods, either turbidimetric or chromogenic. However, both of these methods require higher skill to use and also a machine to read the test, e.g., a microplate reader. The test kit of the present invention combines the ease of use of the gel-clot assay with the speed and variable sensitivity of the photometric methods without the use of specialized equipment or expertise.

In a particularly exemplified test kit according to the invention, the user adds 0.5 mL of sample (e.g., water or dialysate) directly to the first container (e.g., test tube) marked “Sample.” Unlike the standard LAL test, there is no need to first reconstitute the LAL with endotoxin-free water—the present test is designed to be reconstituted with the sample itself. The sample is added using the special disposable pipette provided with the kit. Once the “Sample” tube is reconstituted (within 30-60 seconds with slight swirling), one half (0.25 mL) is removed using the same pipette and added to the second container (e.g., test tube) marked “Control.” Thus, the 0.25 mL removed from the first container is added to the second container, which then serves as a “matched” positive control. Using currently available FDA-approved single test vials, it is impossible to internally control the LAL test with a matched positive control since two “different” LAL tubes are needed; one for the test and one for the control. In the test kit described herein, since more than 2 times the volume of LAL is present in the first container, the sample can be split and ultimately controlled, thereby eliminating tube-to-tube variation, pipetting errors, etc., and thus resulting in a more accurate result overall.

After swirling or tapping the second tube, both tubes are placed in a simple block heater or water bath at 37° C.±1° C. and incubated for a period of time dictated by the level of sensitivity desired. Such time periods may be 10, 20, 30, 40, 50, 60, 70, 80, 90, 100, 110, or 120 minutes, or any time in between. The endotoxin content of the positive control is selected according to the sensitivity desired for the kit.

One of the advantages of the claimed invention is the variable sensitivity of the assay. In a preferred embodiment for testing water or dialysate according to the invention, the sensitivity of the “Sample” LAL is 0.25 EU/mL (referred to as lambda). The matched “Control” would contain 2×lambda or 0.5 EU/mL. At this preferred sensitivity, the test would be completed in under 30 minutes-most typically, the test would be completed in approximately 25 minutes. If positive “Controls” containing higher amounts of endotoxin are used, however, the test time will be even shorter; if controls containing lesser amounts of endotoxin are used, the test time will be longer. The sensitivity of the assay may be chosen by the user, and can range from 2.0EU/mL, 1.0 EU/mL, 0.5 EU/mL, 0.25 EU/mL, 0.125 EU/mL, to 0.005 EU/mL of endotoxin (or any amount in between) depending on the time of the test. Accordingly, the timing of the test may vary from approximately 15 minutes when using high concentrations of positive control (e.g., 1.0 EU/mL), to about two hours when using low concentrations of positive control (e.g., 0.005 EU/ml).

The following are provided for exemplification purposes only and are not intended to limit the scope of the invention.

### Preferred Formulation for the Horseshoe Crab Amebocyte Lysate Reagent in the First Container of the Test Kit:

Amebocyte Lysate—derived from Limulus polyphemus; stored frozen in aliquots both for convenience and preservation of activity.
MgSO₄-0.05 M-1.0 M (0.05M is particularly preferred)
NaCI— 1-3% (1% is particularly preferred)
Imidazole.HCI Buffer— 0.020-0.10 mmolar and pH7 (0.025 mmolar and pH 7 is particularly preferred)
Factor G Activation Inhibitor (GI)(described in U.S. Pat. No. 5,641,643)-0.125 mg/mL

### Exemplary Test Kit Instructions for Detecting Endotoxin in an Aqueous Sample:

1. Add 0.5 mL of the sample (e.g., water or dialysate) to one of the LAL reagent tubes (blue cap; tube label recites “SAMPLE”). Use the pipette supplied with the kit and fill to the 0.5 mL mark (near the middle of the pipette there is a ring-mark for 0.5 mL).
2. The contents of the tube should reconstitute rapidly. To ensure complete dissolution, gently mix the contents of the tube by tapping the bottom of the tube lightly several times with your finger.
3. Using the same pipette, remove 0.25 mL (unlabeled ring-mark below 0.5 mL on the pipette) of the liquid from the LAL, blue-capped, tube and transfer the liquid to the positive control tube (red cap; tube label recites “CONTROL”).
4. Gently mix the contents of the tube by lightly tapping the bottom of the tube several times with your finger.
5. Immediately place both tubes in a 37° C. wet or dry bath incubator.
6. Start timing as soon as tubes are placed in the incubator. Note: the incubation time is test kit dependent. See the Certificate of Analysis for the time to be used.
7. When time is up, immediately and carefully remove the tubes one by one from the incubator. Gently invert the tubes until the absence of a solid gel-clot is confirmed or a 180 degree, i.e., complete, inversion is reached. If a solid clot has formed, the test result is positive for endotoxin. If no clot has formed (i.e., the mixture remains liquid, or the clot breaks), the test result is negative for endotoxin.
8. A test is VALID and POSITIVE if the tube labeled “SAMPLE” (original blue capped tube) is scored positive and the tube labeled “CONTROL” (original red capped tube) is scored positive, i.e., both have solid gel-clots. The sample contains >0.25 EU/mL endotoxin.
9. A test is VALID and NEGATIVE if the tube labeled “SAMPLE” (original blue capped tube) is scored negative and the tube labeled “CONTROL” (original red capped tube) is scored positive. The sample contains <0.25 EU/mL endotoxin.
10. A test is INVALID if the tube labeled “CONTROL” (original red capped tube) is scored negative regardless of the results for the tube labeled “SAMPLE.” If this occurs, the technique should be checked and the test should be repeated. If on repeating, the test is still INVALID, the sample undergoing testing would be deemed incompatible for this assay.

Although the foregoing invention has been described in some detail by way of illustration and example for purposes of clarity of understanding, this invention is not limited to the particular embodiments disclosed, but is intended to cover all changes and modifications that are within the spirit and scope of the invention as defined by the appended claims.

All publications and patents mentioned in this specification are indicative of the level of skill of those skilled in the art to which this invention pertains. All publications and patents are herein incorporated by reference to the same extent as if each individual publication or patent application were specifically and individually indicated to be incorporated by reference.

1. A test kit for detecting bacterial endotoxin in an aqueous solution using a gel-clot method, said test kit comprising:
(a) at least one first container containing freeze dried, endotoxin specific, horseshoe crab amebocyte lysate, whereby the sensitivity of the lysate is pre-certified;
(b) at least one second container containing a defined quantity of endotoxin to serve as a positive control, wherein said defined quantity of endotoxin is pre-certified to positively react with the amebocyte lysate present in said first container; and
(c) at least one disposable endotoxin-free transfer instrument.

2. The test kit of claim 1, wherein said horseshoe crab amebocyte lysate in component (a) is from Limulus polyphemus.

3. The test kit of claim 2, wherein said defined quantity of endotoxin in component (b) is two times the sensitivity of the amebocyte lysate in component (a).

4. The test kit of claim 3, wherein the level of sensitivity of the test kit for detecting endotoxin can vary based on the formulation of the amebocyte lysate in container one and the incubation time of containers one and two.

5. The test kit of claim 2, wherein the amount of said amebocyte lysate is 0.4, 0.5, or 0.6 mL.

6. The test kit of claim 5, wherein the amount of said amebocyte lysate is 0.5 mL.

7. The test kit of claim 2, wherein said aqueous solution is purified, distilled, sterile, non-sterile, or filtered water, water for injection, water for irrigation, or reverse osmosis water.

8. The test kit of claim 2, wherein said aqueous solution is dialysate.

9. The test kit of claim 2, wherein said first and second containers are test tubes.

10. The test kit of claim 9, wherein said test tubes are 12×75 mm and round-bottomed.

11. The test kit of claim 2, where said disposable endotoxin-free transfer instrument is a pipette.

12. The test kit of claim 2, further comprising written instructions for carrying out the test.

13. The test kit of claims 2, further comprising a written certificate of analysis of the amebocyte lysate sensitivity, the quantity of endotoxin in the positive control, and/or the endotoxin-free nature of the transfer instrument.

14. In a method for specifically detecting bacterial endotoxin in an aqueous solution by a gel clot method using horseshoe crab amebocyte lysate, the improvement comprising using a defined quantity of endotoxin to serve as a positive control, wherein said defined quantity of endotoxin is pre-certified to positively react with the horseshoe crab amebocyte lysate, and whereby the sensitivity of said gel clot method can vary based on the time of incubation of the test.

15. The method of claim 14, wherein said horseshoe crab amebocyte lysate is from Limulus polyphemus.

16. The method of claim 15, wherein the sensitivity of said gel clot method can vary based on the formulation of the amebocyte lysate.

17. A method of detecting endotoxin comprising:
(a) reconstituting freeze dried, endotoxin specific, horseshoe crab amebocyte lysate in a first container by adding sample to the first container, whereby the sensitivity of the horseshoe crab amebocyte lysate is pre-certified; and
(b) adding about one-half of contents from the first container to a second container comprising a defined quantity of endotoxin to serve as a positive control, wherein said defined quantity of endotoxin is pre-certified to positively react with the horseshoe crab amebocyte lysate present in the first container.

18. A kit for detecting endotoxin, the kit configured with a defined quantity of endotoxin to serve as a positive control, wherein said defined quantity of endotoxin is pre-certified to positively react with horseshoe crab amebocyte lysate, and whereby the sensitivity of the kit can vary based on the time of incubation of the test.
